(19) United States
(12) Patent Application Publication
(10) Pub. No.: US 2015/0087883 A1
(43) Pub. Date: Mar. 26, 2015

(54) TREATMENT THEATER FOR PROTON THERAPY

(71) Applicant: ProNova Solutions, LLC, Knoxville, TN (US)

(72) Inventors: Cory R. Boudreau, Lake Elmo, MN (US); Stephen A. Latham, Sun Prairie, WI (US); Jarod Beukelman, Lake Mills, WI (US); Niek Schreuder, Knoxville, TN (US); Joseph C. Matteo, Walland, TN (US); Jonathan Huber, Knoxville, TN (US); William Hansen, Lakeville, MN (US)

(21) Appl. No.: 14/492,821

(22) Filed: Sep. 22, 2014

Related U.S. Application Data
(60) Provisional application No. 61/880,423, filed on Sep. 20, 2013.

(51) Int. Cl. A61N 5/10

(52) U.S. Cl. CPC A61N 5/1081; A61N 5/1049; A61N 2005/1087
USPC 600/1

(57) ABSTRACT

A theater system for a proton treatment system, including a tunnel structure configured around an isocenter of a proton treatment system, and one or more devices mounted on the tunnel structure to communicate with a patient located on a treatment bed of the proton treatment system.

### CROSS-REFERENCE TO RELATED APPLICATION

This application claims the benefit of U.S. Provisional Patent Application Ser. No. 61/880,423, filed on Sep. 20, 2013, the disclosure of which is incorporated by reference herein in its entirety.

### FIELD OF INVENTION

The present general inventive concept relates to proton therapy for cancer treatment, and more particularly, to a treatment room theater system suitable for use with proton delivery treatment systems.

### BACKGROUND

Proton Therapy (PT) is a cancer treatment technology that uses high energy protons to penetrate a patient's body and deposit energy into treatment areas such as cancerous tumors. PT leverages the Bragg peak property of charged particles, such as protons, to deposit the majority of the particle's energy in the last few millimeters of travel, as opposed to conventional radiation therapy where the majority of energy is deposited in the first few millimeters of travel—which often causes significant damage to healthy tissue.

PT systems commonly implement a rotating gantry wheel to direct a beam of protons into the patient through a proton delivery nozzle from various positions around the patient during the course of treatment. The beam of protons directed into the patient is targeted into the three-dimensional shape of the desired treatment volume to deliver the therapeutic radiation precisely to the targeted location, while sparing the surrounding healthy tissue. The patient is located on a table and the proton delivery system is located on a rotating structure that may rotate around the patient to allow for delivery of the proton beam from many angles in the plane of rotation.

Patient positioning and treatment involve numerous pieces of equipment that may move about the treatment room including the patient positioning system, the proton beam delivery system, and various forms of imaging equipment.

Once the patient is properly positioned, the patient will be situated within a confined treatment room throughout the treatment process. A course of radiation therapy can run from several minutes up to hours each day, with a typical radiation therapy session lasting from 15 minutes to several hours. A patient's time spent immobilized in a confined space during the treatment process can create discomfort, which may interfere with a patient's ability to maintain still in the proper position throughout the treatment process.

The ability to relax a patient and communicate with a patient during the treatment process can improve the effectiveness of the treatment. The design and arrangement of conventional proton treatment systems makes it difficult to integrate additional audio/visual components such as integrated lasers, speakers, intercoms, tracking devices, projectors, displays, and the like, in order to create a theater-type system which is accessible by a patient at various positions with respect to the confined proton treatment room.

### BRIEF SUMMARY

Example embodiments of the present general inventive concept provide a treatment theater for proton therapy that simplifies the traditional designs of the radiation therapy system and provides changes in the treatment environment that improves the treatment experience for the patient. In some embodiments, the present general inventive concept provides a tunnel around the treatment isocenter that can be populated with various items and elements to facilitate treatment. The integration of various elements around the isocenter allows a patient to communicate more effectively to the therapist, to receive information pertinent to the treatment process, and to receive an enhanced personalized experience throughout the treatment process, regardless of the position of the patient on the treatment bed.

Example embodiments of the present general inventive concept can be achieved by providing a theater system for a proton treatment system, including a tunnel structure configured around an isocenter of a proton treatment system, and one or more devices mounted on the tunnel structure to communicate with a patient located on a treatment bed of the proton treatment system.

The proton treatment system can include a rotating gantry to rotate a proton delivery nozzle about the patient, the rotating gantry being driven by a track located below the treatment bed.

The tunnel structure can include a back wall defining a front face of the rotating gantry perpendicular to a rotation axis of the gantry, the back wall being configured to remain stationary when the gantry is rotating the proton delivery nozzle about the patient, the back wall configured to receive one or more mounted devices thereon.

The back wall can receive one or more connection members mounted thereto to enable one or more devices mounted to the tunnel structure and/or the back wall to be connected to a source of electrical power.

The proton delivery nozzle can be configured to rotate along a rotational path defined between an overhead section of the tunnel structure and the back wall.

The one or more devices mounted to the tunnel section and/or back wall can be selectively activated, for example by a switching control system, to selectively actuate one or more of the devices for interaction with the patient based on location of the patient relative to the tunnel structure and/or based on location of the proton delivery nozzle relative to the patient.

Additional features and embodiments of the present general inventive concept will be set forth in part in the description which follows, and, in part, will be obvious from the description, or may be learned by practice of the present general inventive concept.

### BRIEF DESCRIPTION OF THE DRAWINGS

The following example embodiments are representative of example techniques and structures designed to carry out objectives of the present general inventive concept, but the present general inventive concept is not limited to these example embodiments. In the accompanying drawings and illustrations, the sizes and relative sizes, shapes, and qualities of lines, entities, and regions may be exaggerated for clarity. A wide variety of additional embodiments will be more readily understood and appreciated through the following detailed description of the example embodiments, with reference to the accompanying drawings in which:

FIGS. 1A to 1C are perspective views illustrating a proton treatment theater system configured in accordance with example embodiments of the present general inventive concept;

FIG. 2 is an overhead cross-sectional perspective view of a proton treatment room configured in accordance with an example embodiment of the present general inventive concept;

FIG. 3 is a graphic illustration of a proton therapy treatment room and environment configured in accordance with an example embodiment of the present general inventive concept;

FIG. 4 is a close up of a portion of the graphic illustration of FIG. 3, of a proton therapy treatment room and environment configured in accordance with an example embodiment of the present general inventive concept;

FIG. 5 is a top plan graphic illustration of a proton therapy treatment room and environment configured in accordance with an example embodiment of the present general inventive concept; and

FIG. 6 is a graphic illustration of another embodiment proton therapy treatment room and environment, having a fixed nozzle, configured in accordance with an example embodiment of the present general inventive concept;

### DETAILED DESCRIPTION

Reference will now be made to the following example embodiments of the present general inventive concept, examples of which are illustrated in the accompanying drawings. The example embodiments are described herein in order to explain the present general inventive concept by referring to the figures.

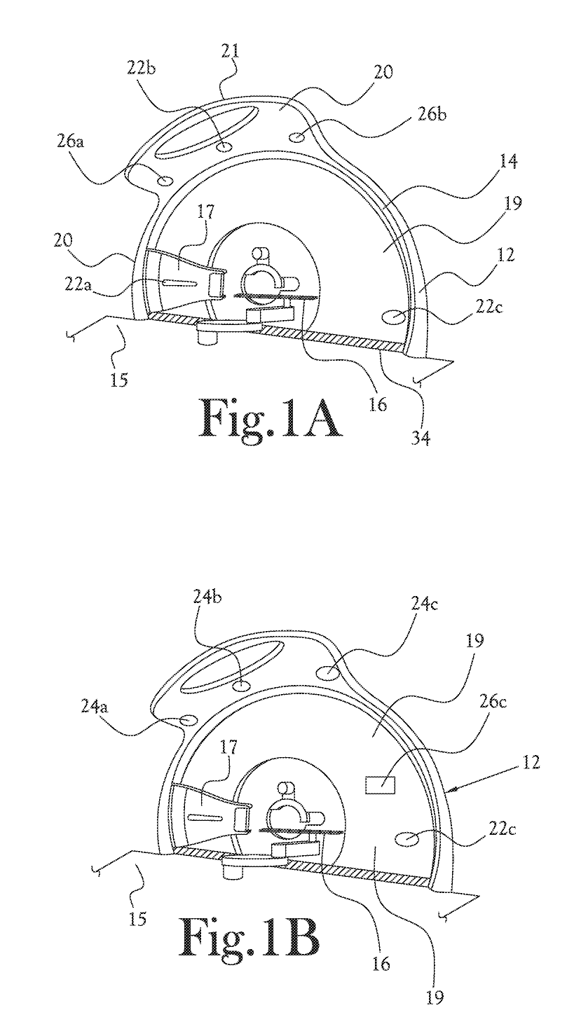

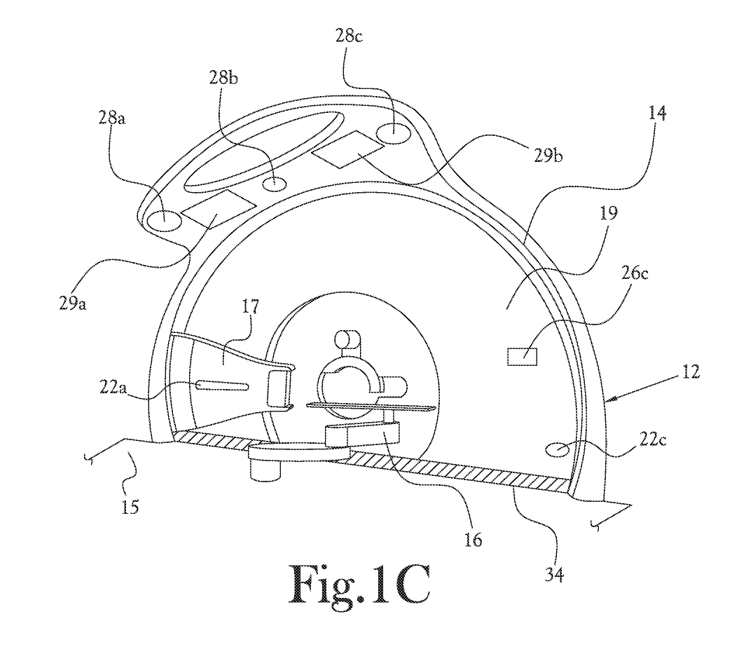

FIGS. 1A to 1C are perspective views illustrating a proton treatment theater system 10 configured in accordance with example embodiments of the present general inventive concept. Referring to FIGS. 1A to 1C, the theater system 10 includes a tunnel 12 around the treatment isocenter that can be populated with various items and elements to facilitate treatment (e.g. projectors, microphones, speakers, lasers, etc). The tunnel may extend out into the room to provide further opportunities for mounting components as well as providing an area for projection of further information, images, entertainment or use of lighting for different effects to be viewed by the patient.

The rotating gantry system 18, sometimes referred to as a gantry wheel, used in connection with the present general inventive concept is coupled to a track which is located below the treatment bed 16, enabling the nozzle 17 to rotate above and below the patient without causing movement of the fixed back wall 19 or tunnel 12. By comparison, conventional rotating gantry products have a moving track situated around the tunnel area, making it impossible to implement a tunnel structure incorporating a variety of functions, as illustrated herein. In this regard, as more fully described in co-pending, and co-owned, patent application Ser. No. 14/064,711, in an example embodiment of a proton therapy (PT) theater 10 a gantry wheel 18 rotates the nozzle 17 from an angle between zero and 360 in order to direct a proton beam through the nozzle 17 toward a patient lying on a treatment bed 16 near the isocenter of the gantry wheel 18. The gantry system 18 may include a mezzanine platform and may include an active (or moving) floor 34 for a technician to walk on, enabling a technician to access magnets, nozzles, achromat and hoses from a beamline and cooling system, etc. for service or replacement. The active floor 34 has an opening provided therein for providing clearance for the nozzle 17 when the nozzle 17 is rotated underneath the patient.

Due to the unique construction of the present rotating gantry 18, many of the mounted components are more readily integrated with the treatment system because of their mounting in the functional tunnel 12 or on the fixed back wall 19. For example, the tunnel structure can include a back wall 19 to defines a planar front face of the rotating gantry perpendicular to a rotation axis of the gantry. The back wall can include a perimeter surface concentric with an overhead section of the tunnel structure. Since the back wall is configured to remain stationary or fixed when the gantry is rotating the proton delivery nozzle about the patient, the fixed back wall 19 can receive one or more mounted devices thereon, as well as connection members such as wires and circuits or other hardware members used to connect one or mounted devices to a source of electrical power. As examples of integration, the loudspeakers can be directional to the patient. Directional loudspeakers limit the volume setting necessary for the patent to hear. It is customary to play music to help the patient relax; the change to directional loudspeakers limits the negative impact of loud music on treatment activities. Further, the same loudspeakers are also a portion of the room intercom and the intercom controller also controls the music, thus the music can have its volume diminished when the intercom is active. The intercom microphone to the patient can similarly be directional.

Another example of integration can be achieved by replacing customary video monitors on the wall with a projected image. Such a change has the advantage of being more adaptable to variations in the installation from one site to another. Use of the projector is made possible by the functional tunnel, which can be selectively configured to allow the patient to view projections from a number of different patient orientations.

Integrating a variety of functions supportive of treatment using embodiments of the present general inventive concept simplifies design of the radiation therapy system, simplifies how the radiation therapy system is used, and provides changes in the treatment environment which improve the treatment experience for the patient.

In some embodiments, the tunnel 12 may be provided adjacent to a radiation treatment device to enable mounting and integration of various functional and entertainment components to facilitate treatment and provide entertainment to the patient, thereby allowing changes in the treatment environment which improves the overall treatment experience for the patient.

Referring to FIGS. 1A to 1C, the tunnel 12 may include a substantially arcuate wall 14 extending from an overhead 20 of the tunnel 12 to the treatment floor 15, but the tunnel 12 is not limited to any particular shape, size, or configuration. In some embodiments the arcuate wall 14 can surround the patient bed 16 and proton delivery nozzle 17 around the perimeter of the back wall 19 to enable the patient to view devices mounted to the wall from multiple orientations on the patient bed 16.

Various devices such as integrated lasers 22a, 22b, 22c, integrated speakers/intercom devices 26a, 26b, 26c, integrated patient movement tracking devices 24a, 24b, 24c, and integrated information projection devices 28a, 28b, 28c may be mounted to the arcuate wall 14 to communicate information regarding the proton therapy to the patient and/or to provide comfort or entertainment of a patient before, during, or after the treatment process. The exact number and placement of devices along the wall 14 and overhead 20 may vary without departing from the spirit and scope of the present general inventive concept. Further, it will be appreciated by those skilled in the art that there are a number of means for mounting media devices, such as speakers, projectors, video monitors, etc. on, or flush with, a vertical or horizontal surface. Any such methods of mounting can be utilized without departing from the spirit or scope of the present general inventive concept.

With regard to lasers 22, as more fully described in co-pending, and co-owned, patent application Ser. No. 14/064,732, the gantry 18 can include alignment and fiducial markers to assist in alignment of the magnets with respect to each other and the gantry 18. For example, in the factory, the magnetic fields can be aligned to the gantry devices, and then fiducial markers can be put on the gantry and the magnets so that when the wheel deflects, triangulation techniques using lasers can be used to pick up the locations of the markers to determine what correction to make to maintain alignment. By being fixed, in relation to the movement of the gantry wheel 18, back wall 19 creates an additional mounting point for integrated lasers 22a, 22b, and 22c or other devices, including connection members to connect the mounted devices to a source of electrical power, not otherwise available with traditional gantry systems which have a rotating back wall.

As illustrated in FIGS. 1A to 1C, the arcuate wall 14 can be shaped to form a generally cylindrical, semi-cylindrical, conical, spheroidal, or semi-spherical shape. However, it will be recognized that other suitable shapes, such as for example ovular, rectangular, triangular, and the like, may be used to define the tunnel without departing from the spirit and scope of the present general inventive concept. In various embodiments, the arcuate wall 14 can include a distal end 21 extending to the proton delivery nozzle 17, but the arcuate wall 14 is not limited to any particular shape or size.

In some embodiments, the tunnel 12 may be configured to receive the mounting of devices suitable to present multimedia outputs to a patient. For example, the tunnel 12 may be configured to present audio media to a patient. An integrated system of one or more speakers and/or intercoms 26a, 26b, 26c, may be configured in the tunnel. The units 26a, 26b, 26c, can be positioned at various locations within the tunnel 12. The speakers may be mounted directional to the patient to enhance the patient treatment experience. Directional speakers limit the volume setting necessary for the patient to hear. During treatment, it is possible to play music to relax the patient, and the flexibility to alter the position of the directional speakers can limit the negative impact of load music on treatment activities. Furthermore, the same speakers can also be a portion of the room intercom, and the control of such intercom system can also control the music. For example, it is possible to configure the speakers/intercoms such that the music can have its volume diminished when the intercom is active. A directional intercom microphone can also be implemented in the units 26a, 26b, 26c, to enable the patient to communicate with a treatment assistant. 9. A switching control system of the integrated system having solid state or other electronic control circuitry can be configured to receive information regarding location of the gantry wheel relative to the patient and/or location of the patient relative to the tunnel structure. The switching control system can then be configured to selectively activate or enable one or more mounted devices for interaction with the patient based on the location of the patient relative to the tunnel structure and/or based on location of the proton delivery nozzle relative to the patient, to enhance the patient's multimedia experience at different viewing angles of the patient and/or operational angles of the proton treatment system.

Referring to FIG. 1C, the tunnel 12 may be configured to include one or more information projection units 28a, 28b, and 28c to display visual media to a patient. The projection units may include one or more screens 29a, 29b mounted along an interior surface of the tunnel 12 to enable the patient to view a particular screen 29a, 29b, depending on the particular patient orientation for a given treatment program. The screen 29a, 29b may be integrally formed with the interior surface of the tunnel 12, and the projection of the visual media may be directional to the patient, enabling the information, image, videos or other projections to be cast at various locations within the tunnel 12. The integration of projections at various locations within the tunnel 12 allows the information and image display more adapted to variations in the installation from one site to another depending on the different positions of the patient during treatment.

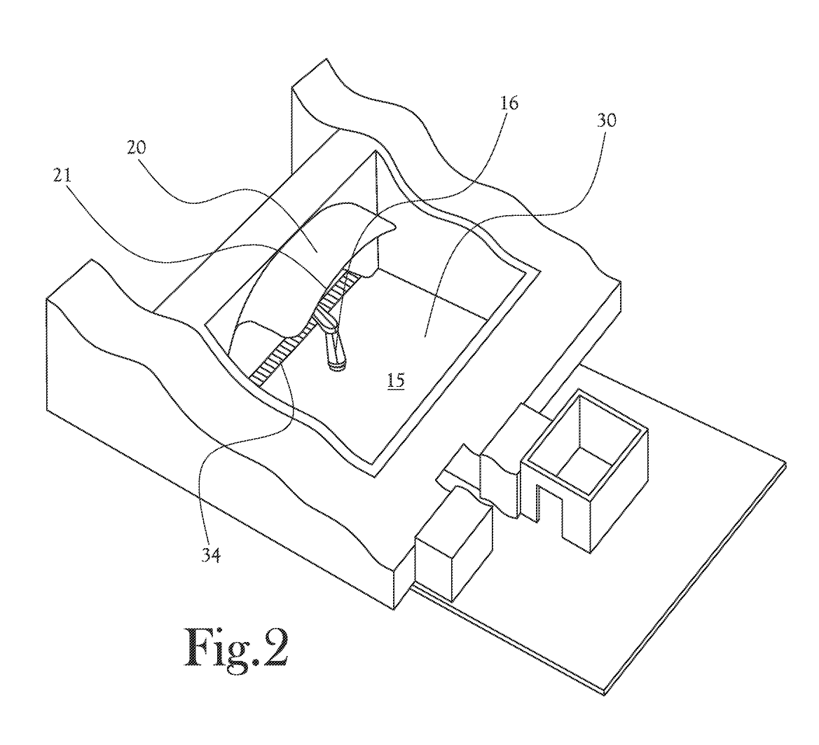

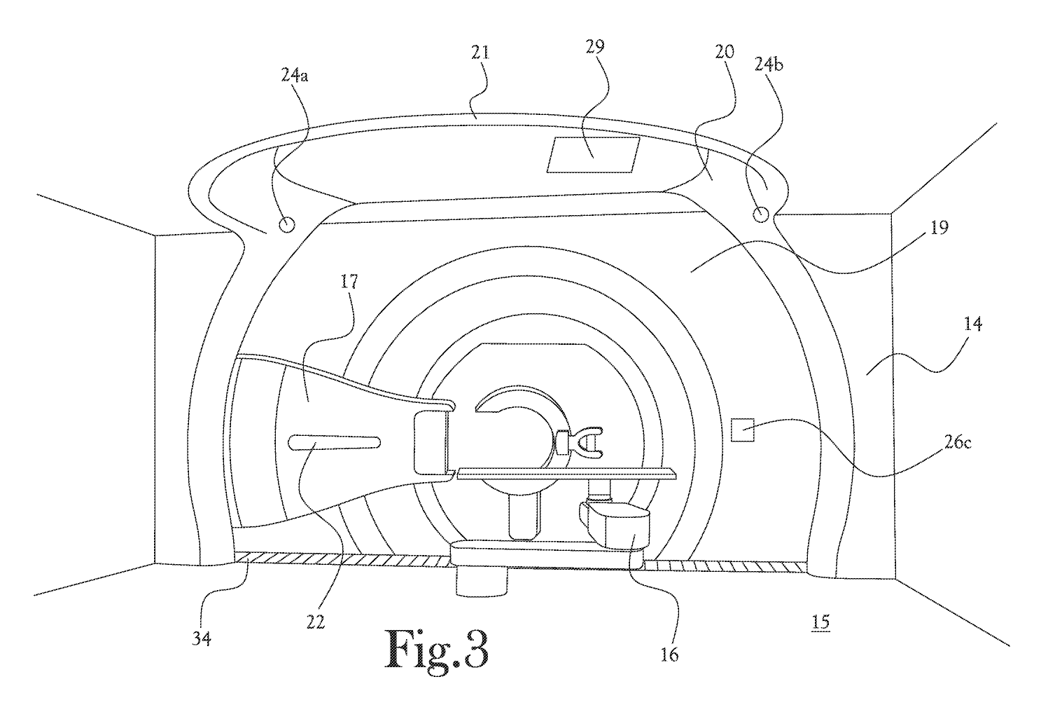

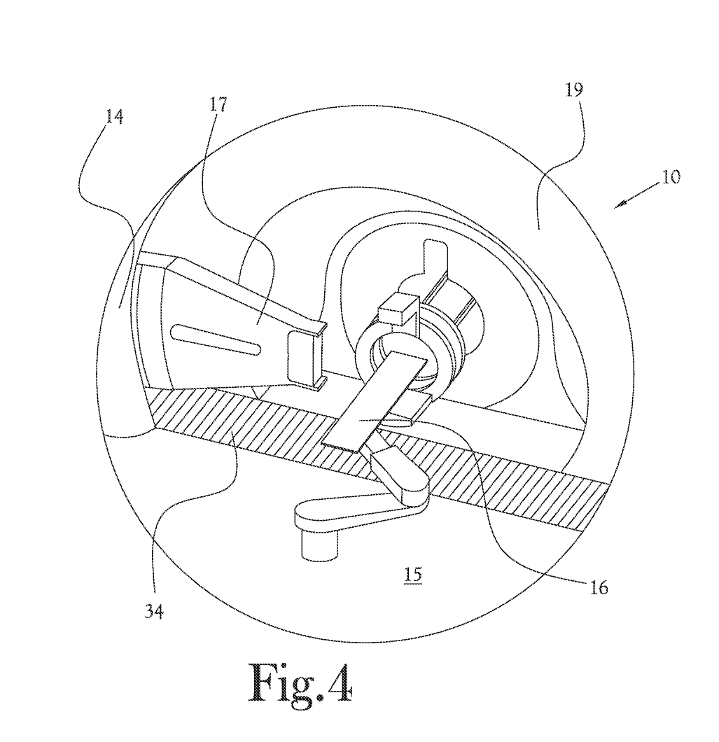

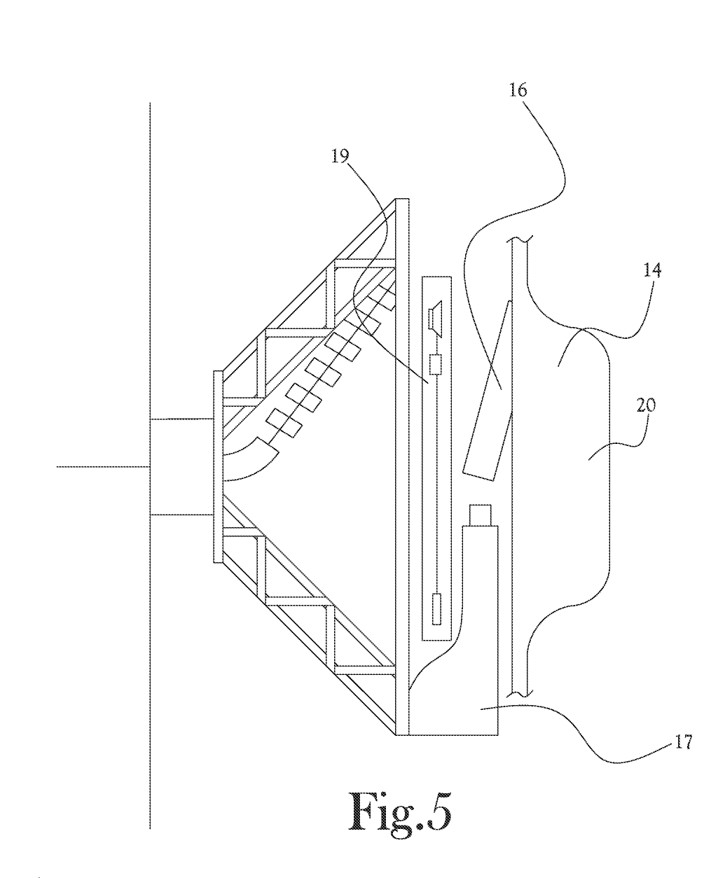

FIG. 2 is an overhead cross-sectional perspective view of a proton treatment room configured in accordance with an example embodiment of the present general inventive concept. As illustrated in FIG. 2, the overhead 20 may be configured to extend out into the treatment room 30 to provide surface area for mounting components as well as providing an area for projection of further information, images, entertainment or use of lighting for different effects to be viewed by the patient.

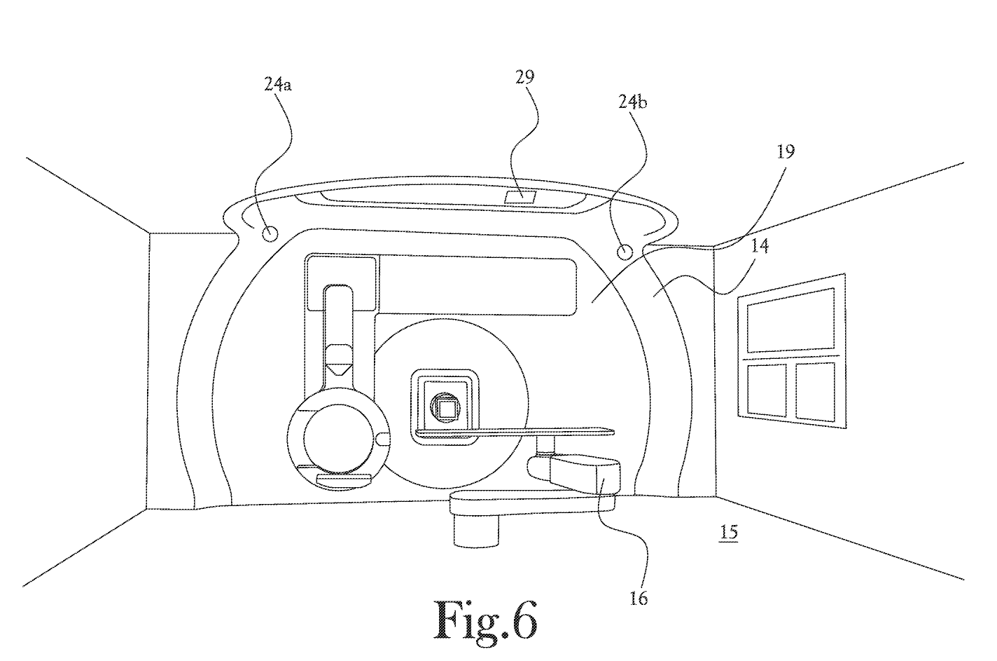

While the treatment theater 10, inclusive of the tunnel 12, and the arcuate wall 14 has been described within the context of a treatment room using a rotatable nozzle 17, it will be appreciated that in another embodiment, the treatment theater of the present general inventive concept could also be adapted for use in a proton therapy treatment room that utilizes a fixed nozzle, as illustrated in FIG. 6.

Embodiments of the present general inventive concept provide a proton treatment room theater. The theater can take the form of a tunnel structure extending around the treatment isocenter. The tunnel can be populated with various audio/visual devices, sensors, trackers, etc., to facilitate treatment and provide entertainment to the patient throughout the treatment process. The tunnel allows for directionality and adaptability of the various placements of the functional and entertainment components and or devices. The present general inventive concept may be used, for example, at research centers, proton therapy treatment providers or in any of other radiation therapy treatment facilities.

Example embodiments of the present general inventive concept can also be achieved by providing a theater system for use in a proton treatments system for a patient, wherein the proton treatment system includes a rotating gantry for rotating a proton delivery nozzle about a patient, the theater system including a fixed wall member proximate the rotating gantry and configured to maintain one or more devices at a fixed location relative to the patient when the gantry is rotated, a tunnel structure configured around an isocenter of a proton treatment system, wherein the tunnel structure includes an arcuate wall member extending around the isocenter, wherein the tunnel structure is configured to maintain the one or more devices at a fixed location relative to the patient when the gantry is rotating the proton delivery nozzle about the patient.

The arcuate wall can be disposed substantially concentric with a rotational path of a proton delivery nozzle of the proton treatment system.

The fixed wall member and the tunnel structure can be disposed in relation to one another such that the proton delivery nozzle is disposed between the fixed wall member and the tunnel structure.

The tunnel can include an overhead section extending over the treatment bed.

One or more devices including one or more of integrated lasers, integrated speakers, integrated intercoms, integrated patient movement tracking devices, and/or an integrated information projection devices.

Example embodiments of the present general inventive concept can also be achieved by providing a theatre system for use in a proton treatment system, wherein the proton treatment system includes a rotating gantry for rotating a proton delivery nozzle about a patient, the theater system including a fixed wall member proximate the rotating gantry and configured to maintain one or more devices at a fixed location relative to the patient when the gantry is rotated, a tunnel structure configured around an isocenter of a proton treatment system, wherein the tunnel structure includes an arcuate wall member extending around the isocenter and an overhead section extending over the treatment bed, wherein the tunnel structure is configured to maintain the one or more devices at a fixed location relative to the patient when the gantry is rotating the proton delivery nozzle about the patient.

While the present general inventive concept has been illustrated by description of several embodiments and while the illustrative embodiments have been described in considerable detail, it is not the intention of the applicant to restrict or in any way limit the scope of the appended claims to such detail. Additional features and embodiments will readily appear to those skilled in the art. The invention in its broader aspects is therefore not limited to the specific details, representative apparatus and methods, and illustrative examples shown and described. Accordingly, departures may be made from such details without departing from the spirit or scope of applicant's general inventive concept.

What is claimed is:

1. A theater system for a proton treatment system, comprising:
a tunnel structure configured around an isocenter of a proton treatment system; and
one or more devices mounted on the tunnel structure to communicate with a patient located on a treatment bed of the proton treatment system.

2. The theater system of claim 1, wherein the proton treatment system includes a rotating gantry to rotate a proton delivery nozzle about the patient, the rotating gantry being driven by a track located below the treatment bed.

3. The theater system of claim 2, wherein the tunnel structure includes a arcuate back wall defining a front face of the rotating gantry perpendicular to a rotation axis of the gantry, the back wall configured to remain stationary when the gantry is rotating the proton delivery nozzle about the patient, the back wall configured to receive one or more mounted devices thereon.

4. The theater system of claim 2, further comprising connection members mounted to the back wall to connect one or more devices mounted to the tunnel structure to a source of electrical power.

5. The theatre system of claim 3, wherein the proton delivery nozzle is configured to rotate along a rotational path defined between an overhead section of the tunnel structure and the back wall.

6. The theater system of claim 1, wherein the tunnel includes an overhead section extending over the treatment bed concentric with a perimeter of the back wall.

8. The theater system of claim 1, wherein the one or more devices include one or more of an integrated laser, an integrated speaker, a display screen, an integrated intercom, an integrated patient movement tracking device, and/or an integrated information projection device.

9. The theater system of claim 8, further comprising a switching controller to selectively activate one or more devices for interaction with the patient based on location of the patient relative to the tunnel structure.

10. The theater system of claim 8, further comprising a switching controller to selectively activate one or more devices for interaction with the patient based on location of the proton delivery nozzle relative to the patient.

11. A theater system for use in a proton treatment system for a patient, wherein the proton treatment system includes a rotating gantry for rotating a proton delivery nozzle about a patient, the theater system comprising:
a tunnel structure configured around an isocenter of a proton treatment system, the tunnel structure having an overhead section for mounting one or more devices thereon such that the one or more devices remain at a fixed location relative to the patient when the gantry is rotating the proton delivery nozzle about the patient; and
a fixed wall member defining a front face of the rotating gantry, the fixed wall member being configured to remain stationary when the gantry rotates the proton delivery nozzle about the patient, the fixed wall member being configured to receive connection members to connect the one or more devices mounted to the tunnel structure to a source of electrical power.

12. The theater system of claim 11, further comprising an arcuate wall disposed substantially concentric with a rotational path of a proton delivery nozzle of the proton treatment system to receive one or more devices thereon.

13. The theater system of claim 11, wherein the fixed wall member and the tunnel structure are disposed in relation to one another such that the proton delivery nozzle is disposed between the fixed wall member and the tunnel structure.

14. The theater system of claim 11, wherein the one or more devices include one or more of an integrated laser, an integrated speaker, an integrated intercom, an integrated patient movement tracking device, and/or an integrated information projection device.
